(19) United States
(12) Patent Application Publication
(10) Pub. No.: US 2026/0250220 A1
(43) Pub. Date: Aug. 27, 2026

(54) PRODUCTION OF ETHYLENE VIA OXIDATIVE DEHYDROGENATION OF ETHANE

(71) Applicant: SABIC GLOBAL TECHNOLOGIES B.V., BERGEN OP ZOOM (NL)

(72) Inventors: Zheng LIU, Bangalore (IN); Robert Roger BROEKHUIS, Geleen (NL); Tian GU, Geleen (NL); Muralikrishnan GOPALAKRISHNAN, Geleen (NL)

(73) Assignee: SABIC GLOBAL TECHNOLOGIES B.V., BERGEN OP ZOOM (NL)

(21) Appl. No.: 18/877,904

(22) PCT Filed: Jun. 7, 2023
(86) PCT No.: PCT/EP2023/065232

§ 371 Date: Dec. 20, 2024

(30) Foreign Application Priority Data
Jun. 23, 2022 (EP) 22180711.8

(51) Int. Cl. C07C 5/48; C07C 11/04

(52) U.S. Cl. CPC C07C 5/48; C07C 11/04; C07C 2523/28

(57) ABSTRACT

Processes for producing ethylene from ethane are described. A process can include oxidatively dehydrogenating ethane to produce a product stream that includes ethylene, ethane, and gases having a lower boiling temperature than ethylene. The product stream can be cooled under mild conditions and then separated to produce a stream that includes ethylene and ethane and a stream that includes the gases having a lower boiling temperature than ethylene.

### CROSS REFERENCE TO RELATED APPLICATIONS

None.

### BACKGROUND OF THE INVENTION

### A. Field of the Invention

The invention generally concerns production of ethylene through oxidative dehydrogenation of ethane. A stream containing ethylene and light gases resulting from the oxidatively dehydrogenation process can be cooled under mild conditions and then separated to produce a stream that includes ethylene.

### B. Description of Related Art

Olefins (e.g., ethylene), are basic building blocks for a variety of commercially valuable polymers. Since naturally occurring sources of olefins do not exist in commercial quantities polymer producers rely on methods for converting the more abundant lower alkanes into olefins. The method of choice for today's commercial scale producers is steam cracking, a highly endothermic process where steam-diluted alkanes are subjected briefly to a temperature of at least 800° C. The fuel demand to produce the required temperatures and the need for equipment that can withstand that temperature add significantly to the overall cost. Also, the high temperature promotes the formation of coke which accumulates within the system, resulting in the need for costly periodic reactor shut-down for maintenance and coke removal.

Oxidative dehydrogenation (ODH) is an alternative to steam cracking. It is an exothermic reaction and produces little or no coke. Examples of ODH processes are described in U.S. Patent Application Publication No. 2020/0407289 to Simazhenkov et al. titled “Mitigating oxygen, carbon dioxide and/or acetylene output from an ODH process”, International Application Publication No. WO2017046315 to Renée et. al. titled “Alkane oxidative dehydrogenation”, and International Application No. WO2015113747 to Winkler et. al., titled “Dilution of the reactants of an oxidative dehydrogenation of alkanes with carbon dioxide”. These processes suffer in that they use high amounts of methane, carbon dioxide, and/or nitrogen as a diluent, which can require more energy to be expended in the separation process. The removal of such compounds and/or other light gases is described in many prior art references, for example WO 2010/115108 to Stephen et al., titled “Process for producing ethylene via oxidative dehydrogenation (ODH) of ethane”. Such processes employ extensive compression and significantly low temperature in the chilling unit before cryogenic separation in order to liquify streams in order to remove by-products and produce ethylene, which can require more energy to be expended in the separation process.

Overall, while the technologies of producing olefins, namely ethylene, exist, they can be energy inefficient and expensive.

### SUMMARY OF THE INVENTION

A discovery has been made that provides a solution to at least one of the problems associated with oxidative dehydrogenation of ethane. In one aspect, the discovery can include a process that allows for cooling a first product stream obtained from an oxidative dehydrogenation process (ODH) without requiring compression. The first product stream can include ethane, ethylene, and one or more gases having a lower boiling temperature than ethylene. The process can include cooling the first product stream to a temperature of from −40° C. to −10° C. and optionally maintaining the pressure of the stream at or near the pressure of the ODH outlet. Ethylene and ethane can then be separated from the first product stream to form a second product stream containing ethylene and ethane, which can be further processed to produce an ethylene stream. The process of the present invention provides the advantage of removing the need for compressing the ethylene-containing stream(s) from the ODH reactor to produce conditions that allow separation of the product stream prior to distillation. This allows for a more efficient and economically feasible process for the production of ethylene when compared with known processes such as those described above. The process of the present invention provides an advantage of reducing or removing the need for compressing the stream to produce conditions that allow separation of the first product stream. Compression can be avoided due to one or more of the following advantageous aspects of the inventive process: (1) diluents are not introduced into the ODH reactor; (2) relatively small amounts of light byproducts are formed due to operation at limited conversion and high selectivity; and (3) the ODH reactor is operated at high pressure. This allows for a more efficient and economically attractive process for the production of ethylene when compared with known processes such as those described above.

In one aspect of the present invention, ODH processes for converting ethane to ethylene are described. One process can include one or more processing steps, such as steps (a), (b), and (c). In step (a), a feed stream that includes ethane can be contacted with an oxidative dehydrogenation catalyst in the presence of oxygen under conditions sufficient to produce a first product stream. ODH conditions can include acceptable temperature and pressure conditions. In one aspect, the pressure can range from 1.8 MPa to 5 MPa, preferably 2 MPa to 4 MPa, more preferably 2 MPa to 3 MPa, or most preferably 2 MPa to 2.5 MPa. In one aspect, the temperature can range from 200° C. to 450° C., preferably 250° C. to 350° C. In some particular aspects, the temperature can be 200° C. to 450° C., preferably 250° C. to 350° C., at an inlet of an ODH reactor. Ethane conversion can range from 20% to 40%. The first product stream can include ethane, ethylene, and one or more gases having a lower boiling temperature than ethylene. Gases having a lower boiling temperature than ethylene can include methane, nitrogen, argon, carbon monoxide, or oxygen, or any combination of or all of said gases. The combined mole fraction of these gases in the first product stream is below 3 percent. In one aspect, the first product stream can be subjected to conditions suitable to oxidize the carbon monoxide to carbon dioxide. The carbon dioxide can be separated from the first product stream downstream of the ODH unit. In one aspect, methane is not added to the feed stream of step (a). In some aspects of the present invention, the first product stream can also include water and acetic acid. In some aspects, a portion of the water and the acetic acid can be removed from the first product stream, for example, by intermediate cooling and gas-liquid separation, prior to cooling the first product stream.

Step (b) of a process of the present invention can include reducing the temperature of the first product stream to a temperature of −40° C. to −10° C., preferably −30° C. to −10° C. The pressure of the stream can be the same or slightly less than the pressure of an outlet of the ODH unit, for example, the last ODH reactor in the ODH unit. For example, 1.5 MPa to 4.5 MPa, preferably 2 MPa to 3 MPa, more preferably 2 MPa to 3 MPa, or most preferably 2 MPa to 2.5 MPa. Step (b) can also include feeding the first product to an inlet of a gas separation unit (e.g., a demethanizer unit) at a temperature of −40° C. to −10° C., preferably −30° C. to −20° C. The first product stream is, in some aspects, preferably in a liquid phase after cooling. Advantageously, the first product stream does not require compression to enter the gas separation unit and/or cool the first product stream to −40° C. to −10° C. This can help increase the energy and cost efficiencies of the overall ODH process.

Step (c) of a process of the present invention can include subjecting the cooled first product stream of step (b) to conditions sufficient (e.g., in a demethanizer) to separate the one or more gases having a lower boiling temperature than ethylene from the cooled first product stream to produce a second product stream comprising ethane and ethylene. Further processing of the second product stream can include separating the second product stream into an ethylene product stream and an ethane stream. In some aspects, the ethane can be recycled to the feed stream of step (a).

Systems to perform the ODH processes of the present invention are also described. A system can include an oxidative dehydrogenation (ODH) unit, a cooling unit, and a gas separation unit (e.g., a demethanizer unit). These units can be configured to transfer product streams from one unit to another unit (e.g., through conduits, inlets, outlets, valves, etc.). The ODH unit can be capable of producing the first product stream at a pressure of 1.8 MPa to 5 MPa. The cooling unit can be capable of cooling the first product stream to a temperature of −40° C. to −10° C., preferably −30° C. to −20° C. The gas separation unit (e.g., demethanizer unit) can be capable of separating the one or more gases having a lower boiling temperature than ethylene from the first product stream to produce the second product stream that includes ethane and ethylene. The second product stream can have less of the one or more gasses having a lower boiling temperature than ethylene when compared with the first product stream. Advantageously, the system of the present invention does not require a compression unit on the first product stream prior to entering the gas separation unit. The system can further comprise a carbon monoxide oxidizing unit capable of oxidizing any carbon monoxide present in the first product stream to produce carbon dioxide. The carbon monoxide oxidizing unit can be positioned in the oxidative dehydrogenation unit or can be positioned downstream from the oxidative dehydrogenation unit. The system can also include a C2 separation unit capable of separating the second product stream into an ethane stream and an ethylene stream.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to other aspects of the invention. It is contemplated that any embodiment or aspect discussed herein can be combined with other embodiments or aspects discussed herein and/or implemented with respect to any method or composition of the invention, and vice versa. Furthermore, compositions of the invention can be used to achieve methods of the invention.

The following includes definitions of various terms and phrases used throughout this specification.

The phrase “a gas having a lower boiling temperature than ethylene” refers to a gas whose normal boiling temperature is lower than the normal boiling temperature of ethylene, which is −103.7° C.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %”, “vol. %”, or “mol. %” refers to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The processes and systems of the present invention can “comprise,” “consist essentially of,” or “consist of” particular ingredients, components, compositions, etc. disclosed throughout the specification. With respect to the transitional phrase “consisting essentially of,” in one non-limiting aspect, a basic and novel characteristic of the processes and/or systems of the present invention is their abilities to cool a product stream that includes ethane, ethylene, and gases having a lower boiling temperature than ethylene to a temperature of −10° C. to −40° C. under mild conditions and then separating the gases having a lower boiling temperature than ethylene from the product stream to produce a product stream that includes ethane and ethylene. In some aspects, a compression unit is not used on either one or both of the first and second product streams or in the overall ODH reaction and separation processes.

Other objects, features and advantages of the present invention will become apparent from the following FIGURES, detailed description, and examples. It should be understood, however, that the FIGURES, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

Advantages of the present invention may become apparent to those skilled in the art with the benefit of the following detailed description and upon reference to the accompanying drawing.

FIG. 1 is an illustration of an oxidative dehydrogenation system to produce ethylene from ethane.

While the invention is susceptible to various modifications and alternative forms, specific embodiments thereof are shown by way of example in the drawing. The drawing may not be to scale.

### DETAILED DESCRIPTION OF THE INVENTION

A discovery has been made that provides a solution to at least one of the problems associated with oxidative dehydrogenation of alkanes (e.g., ethane). In one aspect, the discovery can include maintaining a pressure of a first product stream generated from the ODH reaction of ethane and cooling the first product stream to temperatures between −40° C. and −10° C. prior to entering a gas separation unit (e.g., a demethanizer) at or similar to a pressure of the ODH unit outlet without compression. This can provide an advantage of removing the need for compressing the stream prior to separation of one or more gases having a lower boiling temperature than ethylene from the first product stream. The processes and systems of the present invention can also mitigate greenhouse gas emissions during the process (e.g., emissions associated with fuel combustion in boilers and/or furnaces), or indirect greenhouse gas emissions associated with the purchase of energy (e.g., electricity or steam). These and other non-limiting aspects of the present invention are discussed in further detail with reference to the Figure.

### A. Oxidative Dehydrogenation of Ethane System and Process

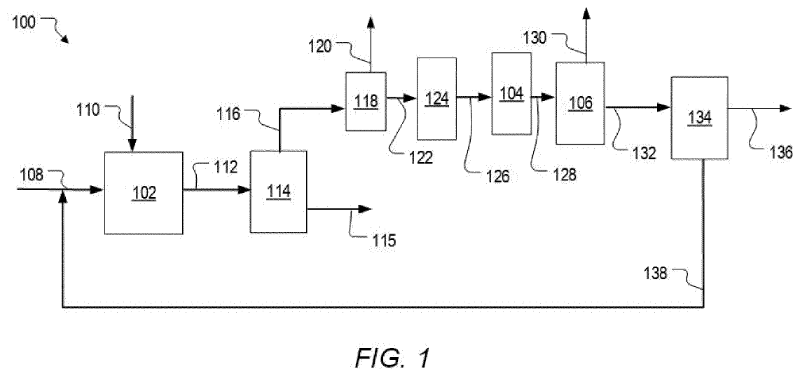

Referring to FIG. 1, a system for the production of ethylene from ethane is described. System 100 can include ODH unit 102, cooling unit 104, and gas separation unit (e.g., a demethanizer) 106 in addition to other auxiliary units.

Ethane feed stream 102 and oxygen-containing stream 108 can enter ODH unit 102. ODH unit 102 can include one or more ODH reactors (e.g., 1, 2, 3, 4, 5, 6, 7, 8, 9, 10 or more). The ODH reactor(s) can be fixed-bed reactor(s) or fluidized-bed reactor(s). Heat can be removed from the ODH reactors or in between/after the reactors. ODH reactor conditions can include temperature and pressure parameters. Pressure in the ODH unit can be from 1.8 MPa to 5 MPa, preferably from 1.9 MPa to 4 MPa, more preferably from 2 MPa to 3 MPa, most preferably from 2 MPa to 2.5 MPa. Non-limiting examples of pressures include 1.8 MPa, 1.9 MPa, 2 MPa, 2.0 MPa, 2.1 MPa, 2.2 MPa, 2.3 MPa, 2.4 MPa, 2.5 MPa, 2.6 MPa, 2.7 MPa, 2.8 MPa, 2.9 MPa, 3.0 MPa, 3.1 MPa, 3.2 MPa, 3.3 MPa, 3.4 MPa, 3.5 MPa, 3.6 MPa, 3.7 MPa, 3.8 MPa, 3.9 MPa, 4.0 MPa, 4.1 MPa, 4.2 MPa, 4.3 MPa, 4.4 MPa, 4.5 MPa, 4.6 MPa, 4.7 MPa, 4.8 MPa, 5.0 MPa and any range or value there between. Temperature in the ODH unit (e.g., ODH reactor(s)) can range from 200° C. to 450° C., preferably from 250° C. and 350° C. Non-limiting examples of temperatures include 200° C., 225° C., 250° C., 275° C., 300° C., 325° C., 350° C., 375° C., 400° C., 425° C., 450° C., and any range or value there between.

In ODH unit 102, ethane and the oxygen-containing feed can mix and contact a dehydrogenation catalyst to produce an ODH product gas stream (first product stream). Catalysts for oxidative dehydrogenation include metals or compounds thereof from Columns 5, 6, 7, 8, 9, 10, and/or 11 of the Periodic Table of Elements and/or any combination thereof. A non-limiting example of the catalyst used in the present invention is Moₐ V_{b}Nb_{c}X_{d}, where X=at least one of the following: Te, Li, Sc, Na, Be, Mg, Ca, Sr, Ba, Ti, Zr, Hf, Y, Ta, Cr, Fe, Co, Ni, Ce, La, Zn, Cd, Hg, Al, Pb, As, Bi, Sb, U, Mn, and W; and a=0.05 to 0.9, b=0.1 to 0.4, c=0.001 to 0.2 and d=0.001 to 1.0. The values of a, b, c and d constitute relative gram-atoms of the elements Mo, V, Nb, and X, respectively. The elements are present in combination with oxygen in the form of various oxides. The catalyst can be supported or non-supported. Suitable supports include silica, aluminum oxide, silicon carbide, zirconia, titania, and mixtures thereof. In principle any catalyst with sufficient activity and selectivity properties which is stable under the given reaction conditions can be used. The catalyst may take the form of a bulk particulate catalyst, a supported particulate catalyst, a supported structured catalyst (for example, a catalytic foam or a monolith), or any other form that promotes the reaction of the gaseous species over the solid catalyst.

ODH product gas stream 112 can include hydrocarbons (e.g., ethane and ethylene) and nonhydrocarbons (e.g., water, acetic acid, argon, nitrogen, carbon oxides, oxygen, and the like).

In some embodiments, ODH unit 102 can include a carbon monoxide oxidation unit that receives the ODH product gas stream. The CO oxidation unit can include one or more CO oxidation reactors. In the CO oxidation unit, CO in the ODH product gas stream can be oxidized to carbon dioxide. Supplemental oxygen can be provided to the CO oxidation reactor to ensure sufficient oxygen is present for the oxidation of CO to CO₂. The CO oxidation unit can be any known unit capable of oxidizing CO to carbon dioxide (CO₂). In some instances, the CO oxidation unit can be placed downstream of ODH unit 102, for example after quenching unit 114.

ODH product gas stream 112 (first product stream) can exit ODH unit 102 and enter quenching unit 114. In quenching unit 114, first product gas stream 112 can be cooled to a temperature sufficiently low, for example 10° C. to 50° C., to condense water and/or acetic acid from the gas stream to produce quenched first product gas stream 116. Water and/or a water acetic acid mixture stream 115 can exit quenching unit 114 and can be further processed, stored or transported. For example, water and acetic acid and be separated and recovered using any known acetic acid/water separation method. Quenching unit 114 can include any equipment capable of liquid/gas separation (e.g., a quench tower or a combination of a cooling exchanger and a gas-liquid separator). Quenched first product gas stream 116 can exit quenching unit 114 and enter carbon dioxide removal unit 118. The pressure of the exiting stream can be the same or slightly less than the pressure of the outlet of the ODH unit. For example, 1.5 MPa to 4.5 MPa, preferably 2 MPa to 3 MPa, more preferably 2 MPa to 3 MPa, or most preferably 1.5 MPa to 1.9 MPa.

Carbon dioxide removal unit 118 can be any known unit capable of separating carbon dioxide from the quenched first product gas stream 116 and form CO₂ stream 120 and first product stream 122 having a lower content of CO₂ as compared to first product gas stream 116 (e.g. a treated first product stream). In some embodiments, carbon dioxide removal unit 118 can be an absorption system (e.g., using amine or carbonate absorbents) or a membrane unit. CO₂ stream 120 can exit CO₂ removal unit 118 and be stored and/or further processed. Advantageously, CO₂ removal unit 118 can be positioned after the CO oxidation unit, allowing it to remove the CO₂ formed by the oxidation of CO. In one embodiment, during the CO, CO₂, water, and/or acetic acid removal processes, the product stream is not compressed prior to feeding the first product stream to the inlet of gas separation unit 106 at a temperature of −40° C. to −10° C.

First product stream 122 can exit CO₂ removal unit 118 and enter drying unit 124. In drying unit 124, first product stream 122 can be subjected to conditions to lower, if desired, the carbon dioxide concentration to parts per billion or parts per million levels and remove all, or substantially all, of any remaining water. Drying unit 124 can be any unit known in the art that is capable of lowering the concentration of water in first product stream 122 and form dried first product stream 126, for instance, an adsorption system based on molecular sieve drying agents. Dried first product stream 126 can exit drying unit 124 and pass through cooling unit 104 to form cooled first product stream 128. Cooling unit 104 can be a heat exchanger rejecting process heat to refrigerant. For example, the first product stream is cooled to about −40° C. to −10° C.-30° C. to −20° C., or −40° C., −35° C., −30° C., −25° C., −20° C., −15° C., −10° C., or any range or value there between through the heat exchange process. After cooling unit 104, the first cooled product stream 128 is in a liquid phase, or in a combination of liquid and gas phases. Notably, no compression of dried first product stream as it is processed from ODH unit 102 to separation unit 106 is needed to condense the first product stream and separate it in separation unit 106 (e.g., a demethanizer). A reason for not needing a compression step is because the pressure of the first product stream can be 1.9 MPa to 5 MPa, preferably 1.9 to 2.3 MPa, during removal of water, carbon dioxide, and one or more gases having a lower boiling temperature than ethylene.

Cooled first product stream 128 can enter separation unit 106 without being further pumped. In an aspect, if cooled first product stream 128 is substantially in the liquid phase it can be pumped to a higher pressure. In an aspect, if cooled first product stream is partially in the vapor phase (e.g., a molar vapor fraction of 1%), the vapor and liquid substreams may be separated in a gas-liquid separator (e.g., a flash drum) directing only the liquid substream towards separation unit 106 (with or without using a pump). In this case, ethane or ethylene carried away in the vapor substream do not contribute to the process yield so process conditions are advantageously chosen so that the molar vapor fraction of the cooled first product stream is below 2%, below 1% or below 0.5%. Separation unit 106 (e.g., a demethanizer unit) can be any known separation unit capable of separating gases having a lower boiling temperature than ethylene from first product stream 128. For example, the separation unit can be a demethanizer unit and/or a distillation unit operated at a temperature in the condenser of −101° C. to −94° C. and pressure of 2.0 MPa to 5 MPa, preferably from 2 MPa to 4 MPa, more preferably from 2 MPa to 3 MPa, most preferably from 2.0 MPa to 2.5 MPa. Non-limiting examples of pressures include, 2.0 MPa, 2.1 MPa, 2.2 MPa, 2.3 MPa, 2.4 MPa, 2.5 MPa, 2.6 MPa, 2.7 MPa, 2.8 MPa, 2.9 MPa, 3.0 MPa, 3.1 MPa, 3.2 MPa, 3.3 MPa, 3.4 MPa, 3.5 MPa, 3.6 MPa, 3.7 MPa, 3.8 MPa, 3.9 MPa, 4.0 MPa, 4.1 MPa, 4.2 MPa, 4.3 MPa, 4.4 MPa, 4.5 MPa, 4.6 MPa, 4.7 MPa, 4.8 MPa, 5.0 MPa and any range or value there between. In separation unit 106, separation of one or more of the gases having a lower boiling temperature than ethylene from first product stream 128 produces gas stream 130 and second product stream 132 that includes ethylene and ethane. Second product stream 132 can exit separation unit 106 and enter C2 separation unit 134 (second gas separation unit). C2 separation unit 134 can be any known separation unit capable of separating ethane from ethylene (e.g., a membrane system and/or a distillation system). In C2 separation unit 134, the second product stream can be separated into ethylene product stream 136 and ethane stream 138. Ethane stream 138 can be recycled to the ODH reactor 102 (e.g., combined with ethane feed stream 108 and/or fed directly into ODH unit 102). Ethylene product stream 136 can exit ethylene separation unit 134 and can be collected, stored, sold, transported, or provided directly to other processing units.

In FIG. 1, units 102, 104, 106, 114, 118, 124, and 134 can include one or more heating and/or cooling devices, and control devices (e.g., valves, flow controllers and related instrumentation), inlets, outlets, etc. that can help control the temperatures and pressures of reactions or separations processes or movement of streams. It should be understood that one or multiple reactors can be housed in one unit. The temperature, pressure, and flowrate can be varied depending on the reaction to be performed and is within the skill of a person performing the reaction (e.g., an engineer or chemist).

### B. Stream Compositions

Ethane gas fed to ODH unit 102 can be obtained from any number of sources, for example from natural gas processing or from intermediate streams in other hydrocarbon processing facilities (e.g., pyrolysis plants), provided that impurities are removed sufficiently to prevent catalyst poisons and eventual product contaminants, and also to avoid economically excessive content of other accompanying compounds, including methane, C3+ hydrocarbons, hydrogen, carbon dioxide, nitrogen, argon, etc. The feed stream to ODH unit 102 may include fresh ethane, such as from sources mentioned above, and may also include recycle of unreacted ethane recovered from the C2 separation unit 134 as shown in FIG. 1.

Oxygen-containing gas fed to ODH unit 102 can include air, oxygen, and/or an air/oxygen mixture. The oxygen-containing gas can be purified oxygen produced by an air separation process. The oxygen-containing gas can contain an inert gas such as nitrogen, helium or argon. In some embodiments, inclusion of some amount of inert gas components may be advantageous for limiting the flammability of some effluent stream(s) that may contain residual oxygen. Preferably, the fraction of components other than oxygen in the oxygen-containing gas is small so that the stream contains at least 90 vol. % at least 95 vol. %, or at least 99 vol. % oxygen.

The feed ratio of ethane to oxygen can be determined to obtain the desired combination of conversions of ethane and oxygen, which are inter-related by material balances and reaction selectivities. The feed components, ethane, oxygen/air, etc. may be vaporized, preheated and mixed to the extent practical prior to feeding to the ODH unit. This can be accomplished by processes known to those skilled in the art (e.g., engineer or chemist). Preheat techniques may include, for example, heat exchange from steam, a heat transfer fluid, reactor effluent, and a furnace. The overall conversion of ethane through the ODH unit may be achieved in a single reactor stage or in a plurality of reactor stages housed in a single or in multiple vessels. In some embodiments, all of the oxygen-containing feed is mixed with the ethane feed stream before entering the ODH unit (i.e., before contacting any catalyst). In other embodiments, the oxygen-containing feed is divided in portions that are fed to various locations before and between reactor stages. The reactor stage(s) may be cooled or adiabatic, and may be configured according to any reactor concepts known in the art. The conversion of ethane through ODH unit increases as the ratio of oxygen-containing gas to ethane feed gas increases, and can be selected to achieve an economically favorable level of conversion, for example, between 10% and 100%, between 15% and 50%, or between 20% and 40%. The conversion of ethane is defined as the fraction of the total molar amount of ethane entering the ODH unit (e.g., fresh ethane and recycled ethane) that is chemically converted into other compounds within the ODH unit.

Various streams may be fed, through separate feed pipes, a combined feed pipe, or a combination thereof, to the ODH unit. Such feed streams may include a fresh ethane feed, a recycled ethane stream, and an oxygen feed. The stream that would result from combining all such feed streams is here referred to as the combined feed stream. The combined feed stream includes ethane and oxygen, and can additionally include low levels of one or more other gases having a lower boiling temperature than ethylene, for example contaminants entering with the ethane or oxygen feed streams. In the inventive process, it is preferred to limit the concentration of gases having a lower boiling temperature than ethylene (e.g., methane, nitrogen, argon, and the like) in the combined feed stream. The combined mole fraction of gases having a lower boiling temperature than ethylene, excluding oxygen, in the combined feed stream entering the ODH unit is below 2 percent or from 0% to 1%, or below 0.5%, or below 2%, 1.75%, 1.5%, 1.25%, 1.0%, 0.75%, 0.5%, 0.25%, 0% or any value or range there between.

First product stream can include ethylene, ethane, and one or more gases having a lower boiling temperature than ethylene. Non-limiting examples of gases having a lower boiling temperature than ethylene include methane, nitrogen, argon, carbon monoxide. The combined mole fraction of the gases having a lower boiling temperature than ethylene in the first product stream can be below 3 percent or from 0% to 2%, or 0.01 to less than 1.5%, or below 0.5%, or 0%, 0.5%, 0.75%, 1%, 1.25%, 1.5%, 1.75%, 2%, 2.25%, 2.5%, 2.75% or any value or range there between. The first product stream can also include carbon dioxide, acetic acid, and/or water.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1

### (ODH of Ethane to Ethylene)

A commercial reactor design was used to build a computer simulation (AspenTech, Bedford, MA) for the process of ethylene production by oxidative dehydrogenation of ethane. Referring to FIG. 1, the ODH unit 102 included 6 adiabatic reactors in series, operated at inlet temperatures from 372° C. to 381 C and 2.1 MPa to 2.3 MPa. The overall ethane conversion was 30% and the ethylene selectivity was about 90%. The ODH unit included a CO oxidation unit to convert most of the CO formed in the ODH reactions to CO₂. Due to the high pressure in the ODH reactor unit 102, there was no need for further compression of the product gas stream from the ODH reactor. In addition, first product stream (stream 128, Table 1) exiting cooling unit 104 is a liquid at −18° C. and 1.9 MPa, which allowed a pump to be used to further increase the pressure of the first product stream to 2.3 MPa prior to feeding the stream into the demethanizer unit (gas separation unit) 106. Information for selected streams in this example according to FIG. 1 is provided in Table 1. Data for mass flows are in kg/hr, temperatures are in ° C. and pressures are in MPa.

TABLE 1

| Stream | 108 | 110 | 112 | 115 | 116 | 120 | 122 |
|---|---|---|---|---|---|---|---|
| Temp | 35 | 35 | 140 | 35 | 35 | 35 | 35 |
| Press. | 2.3 | 2.3 | 2.0 | 2.0 | 2.0 | 2.0 | 2.0 |

Mass Flows

| | | | | | | | |
|---|---|---|---|---|---|---|---|
| N₂ | 0 | 19 | 19 | 0 | 19 | 0 | 19 |
| O₂ | 0 | 204,996 | 704 | 0 | 704 | 0 | 704 |
| Ar | 0 | 460 | 460 | 0 | 460 | 0 | 460 |
| CO | 0 | 0 | 1,196 | 0 | 1,196 | 0 | 1,196 |
| CO₂ | 0 | 0 | 54,505 | 81 | 54,424 | 54,424 | 0 |
| C₂H₄ | 1,202 | 0 | 216,844 | 177 | 216,668 | 0 | 216,668 |
| C₂H₆ | 857,630 | 0 | 600,810 | 699 | 600,111 | 0 | 600,111 |
| H₂O | 0 | 0 | 176,947 | 176,945 | 2 | 0 | 2 |
| AA | 0 | 0 | 12,823 | 12,823 | 0 | 0 | 0 |
| Total | 858,833 | 205,476 | 1,064,309 | 190,724 | 873,585 | 54,424 | 819,161 |

| Stream | 126 | 128 | 130 | 132 | 136 | 138 |
|---|---|---|---|---|---|---|
| Temp | 10 | −17 | −97 | −10 | −31 | −8 |
| Press. | 1.9 | 2.3 | 2.2 | 2.2 | 1.9 | 2.0 |
| Mass Flows | | | | | | |
| N₂ | 19 | 19 | 19 | 0 | 0 | 0 |
| O₂ | 704 | 704 | 611 | 93 | 93 | 0 |
| Ar | 460 | 460 | 407 | 53 | 53 | 0 |
| CO | 1,196 | 1,196 | 1,192 | 4 | 4 | 0 |
| CO₂ | 0 | 0 | 0 | 0 | 0 | 0 |
| C₂H₄ | 216,668 | 216,668 | 155 | 216,513 | 215,311 | 1,202 |
| C₂H₆ | 600,111 | 600,111 | 23 | 600,088 | 108 | 599,981 |
| H₂O | 0 | 0 | 0 | 0 | 0 | 0 |
| AA | 0 | 0 | 0 | 0 | 0 | 0 |
| Total | 819,159 | 819,159 | 2,407 | 816,752 | 215,569 | 601,183 |

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein can be utilized. Accordingly, the appended claims are intended to include within their scope such processes, machines, manufacture, compositions of matter, means, methods, or steps.

1. An oxidative dehydrogenation process for converting ethane to ethylene, the process comprising:
(a) contacting a feed stream comprising ethane with an oxidative dehydrogenation catalyst in the presence of oxygen under conditions sufficient to produce a first product stream comprising ethane, ethylene, and one or more gases having a lower boiling temperature than ethylene, wherein the conditions comprise a pressure of 1.8 MPa to 5 MPa;
(b) feeding the first product stream comprising ethane, ethylene, and one or more gases having a lower boiling temperature than ethylene to an inlet of a gas separation unit at a temperature of −40° C. to −10° C.; and
(c) separating the one or more gases having a lower boiling temperature than ethylene from the first product stream of step (b) to produce a second product stream comprising ethane and ethylene.

2. The process of claim 1, wherein the oxidative dehydrogenation conditions comprise a pressure of 2 MPa to 4 MPa.

3. The process of claim 2, wherein the oxidative dehydrogenation conditions comprise a temperature of 200° C. to 450° C.

4. The process of claim 1, further comprising separating the second product stream into an ethylene product stream and an ethane stream.

5. The process of claim 4, further comprising recycling the ethane stream to step (a).

6. The process of claim 1, wherein the first product stream is not subjected to compression in step (a), step (b), step (c), or a combination thereof.

7. The process of claim 1, wherein the first product stream further comprises water and acetic acid, and the process further comprises, prior to step (b), removing at least a portion of the water and the acetic acid from the first product stream.

8. The process of claim 1, wherein the gases having a lower boiling temperature than ethylene comprise carbon monoxide and the process further comprises removing the carbon monoxide from the first product stream prior to step (b) or subjecting the first product stream to conditions suitable to oxidize the carbon monoxide to carbon dioxide, or a combination thereof.

9. The process of claim 1, wherein the first product stream comprises carbon dioxide and the process further comprises removing the carbon dioxide from the first product stream prior to step (b).

10. The process of claim 1, wherein the ethane conversion in step (a) is between 20% and 40%.

11. The process of claim 1, wherein the first product stream in step (b) is in a liquid phase.

12. The process claim 1, wherein the first product stream in step (b) has a molar vapor fraction smaller than 1%.

13. The process of claim 1, wherein methane is not added to the step (a) feed stream.

14. The process of claim 1, wherein the gases having a lower boiling temperature than ethylene comprise at least one of methane, nitrogen, argon, carbon monoxide, and oxygen, and the combined mole fraction of these gases in the first product stream is below 3%.

15. The process of claim 1, wherein a combined mole fraction of gases having a lower boiling temperature than ethylene, excluding oxygen, of all streams entering an oxygen dehydrogenation unit comprising the oxygen dehydrogenation catalyst of step (a) is below 2%.
